(19) United States
(12) Patent Application Publication
(10) Pub. No.: US 2008/0026463 A1
(43) Pub. Date: Jan. 31, 2008

(54) MATRIX AND METHOD FOR ISOLATION OF HEPATIC PROGENITOR CELLS

(75) Inventors: Joseph Charles Ruiz, Durham, NC (US); Sonya Olabisi Amelia Sherwood, Durham, NC (US); Jennifer C. Clark, Durham, NC (US)

Correspondence Address: FOLEY AND LARDNER LLP;SUITE 500, 3000 K STREET NW, WASHINGTON, DC, 20007, US

(73) Assignee: Vesta Therapeutics, Inc.

(21) Appl. No.: 11/768,452

(22) Filed: Jun. 26, 2007

Related U.S. Application Data
(60) Provisional application No. 60/816,862, filed on Jun. 28, 2006.

(51) Int. Cl. C12N 5/06; C12M 1/00

(52) U.S. Cl. 435/370; 435/307.1

(57) ABSTRACT

A method is provided of isolating and propagating hepatic progenitors in vitro on one or multiple extracellular matrix components comprising a collagen in polymeric form. A container for the isolation and propagation of the progenitors comprising culture dishes and/or bioreactors comprising the disclosed matrices is also provided.

### CROSS-REFERENCE TO RELATED APPLICATIONS

The present application claims priority to U.S. Provisional Application No. 60/816,862, filed Jun. 28, 2006, the disclosure of which is incorporated herein by reference in its entirety.

### BACKGROUND OF THE INVENTION

The present invention relates generally to the ex vivo enrichment and/or isolation and propagation of hepatic progenitor cells. More particularly, the present invention relates to the identification and selection of extracellular matrix components, which enable the propagation and/or isolation of hepatic progenitor cells, including hepatic stem cells, in vitro.

Primary hepatocyte cell transplantation has been tested in clinical studies for safety and efficacy over the last decade as an alternative to whole organ transplantation, demonstrating its potential. Given that the vast majority of hepatocytes isolated from unused donor livers do not have proliferative capabilities, the ability to implant “stem” cell populations that have the ability to form liver tissue in situ would be a major advance in current cell-based therapies. Hepatic stem cells and their progeny (e.g., hepatoblasts and committed progenitors) have considerable expansion potential. For this reason, these cell populations are desirable candidates for cell therapies, including bioartificial livers or cell transplantation, and present an attractive alternative therapeutic option for patients.

Despite this promise, however, the full potential of liver cell therapy remains to be realized. First, isolation of hepatic stem cells can be laborious and time consuming. These cells often comprise less than 5 percent of the total liver cell population, and in many cases less than 1 percent. Current methodologies typically involve positive selection of the target cells using identifiable markers, e.g., antibodies to cell surface proteins. These methods can be costly and, at times, inefficient.

Second, the ex vivo propagation of hepatic stem cells and their progeny has proven to be challenging. Where hepatic stem cells and their progeny are successfully propagated in vitro, the culture conditions are not optimal for transition from the laboratory bench to the clinic. For example, some culture conditions greatly retard cell division or arbitrarily promote cell differentiation, thereby reducing propagation efficacy. As well, some culture conditions require the addition of factors (e.g., serum or feeder cells) that can introduce contaminants and thereby limit their application in treating humans.

Accordingly, there is a need for alternative methods to select for and isolate hepatic progenitor cells. As well, there is a need for culture conditions that can provide enhanced ex vivo propagation of hepatic stem cells and their progeny and a need for conditions that can lineage restrict stem cells into their appropriate fates. Finally, there is a need for culture conditions that obviate the requirement of feeder cells.

### SUMMARY OF THE INVENTION

One embodiment of the invention provides a method of isolating and/or selecting hepatic progenitors in vitro comprising: (a) providing a single cell suspension of hepatic cells; (b) culturing the suspension of hepatic cells on an extracellular matrix comprising a collagen in polymerized form to obtain a population of isolated hepatic progenitor cells. The collagen may be a type I collagen, preferably greater than about 75 percent by weight type I collagen, more preferably greater than about 90 percent by weight type I collagen, and even more preferably greater than about 95 percent by weight type I collagen. In a specific embodiment of the invention, the matrix comprises greater than about 97 percent by weight type I collagen and/or is the commercially available PURECOL matrix. In some embodiments, the matrix may further comprise a type III collagen in polymerized form. As well, hepatic progenitors may be hepatic stem cells. The inventive method may further comprise culturing the suspension of hepatic cells in serum free culture medium.

In another embodiment of the present invention, a composition comprising a cell culture of hepatic progenitor cells, serum-free culture medium, and an extracellular matrix is provided comprising a collagen in polymerized form. The collagen may be a type I collagen, preferably greater than about 75 percent by weight type I collagen, more preferably greater than about 90 percent by weight type I collagen, and even more preferably greater than about 95 percent by weight type I collagen. In a specific embodiment of the invention, the matrix comprises greater than about 97 percent by weight type I collagen and/or is the commercially available PURECOL matrix. In some embodiments, the matrix may further comprises a type III collagen in polymerized form. As well, hepatic progenitors may be hepatic stem cells.

In yet another embodiment of the present invention, a method of propagating hepatic progenitors in vitro is provided comprising: (a) providing a single cell suspension of hepatic cells; (b) culturing the suspension of hepatic cells on an extracellular matrix comprising a collagen and in serum-free culture medium, in which the collagen is in polymerized form. The hepatic progenitors may be hepatic stem cells, isolated hepatoblasts, committed hepatic progenitors, or combinations thereof. Further, the collagen may be a type I collagen, preferably greater than about 75 percent by weight type I collagen, more preferably greater than about 90 percent by weight type I collagen, and even more preferably greater than about 95 percent by weight type I collagen. In a specific embodiment of the invention, the matrix comprises greater than about 97 percent by weight type I collagen and/or is the commercially available PURECOL matrix. In some embodiments, the matrix may further comprise a type III collagen in polymerized form.

In still yet another embodiment of the present invention, a container is provided for propagation of hepatic progenitors comprising a container and an insoluble material comprising at least one collagen in polymerized form, wherein the insoluble material substantially coats at least one surface of the container. The container may be a tissue culture plate, a bioreactor, a lab cell or a lab chip.

### BRIEF DESCRIPTION OF THE DRAWINGS

FIG. 1 shows the morphology of stem cell colonies derived from neonatal liver two weeks post isolation on an inventive matrix according to the invention.

FIG. 2 shows that the colonies of FIG. 1 are EpCAM positive (marker for hepatic stem cells).

FIG. 3 shows the morphology of stem cell colonies derived from fetal liver two weeks post isolation on an inventive matrix according to the invention.

FIG. 4 shows RNA expression of select markers from cells grown on a PureCol™ matrix according to the invention. cDNA was generated from cell lysates and then subject to PCR for the following markers: Lanes: 1, EPCAM; 2, Albumin; 3, Alpha-fetoprotein; 4, CK19; 5, CYP3A4; 6, Transferrin; 7, alpha-1-antitrypsin; 8, Dipeptylpeptidase 4; 9, Aquaporin 4; 10, GAPDH.

### DETAILED DESCRIPTION OF EMBODIMENTS OF THE INVENTION

In one embodiment of the present invention, extracellular matrix components have been identified, which facilitate the attachment, survival and ex vivo proliferation of hepatic stem cells and their progeny. The term “hepatic progenitors,” as used herein, is broadly defined to encompass both hepatic stem cells and their progeny. “Progeny” may include both self-replicating hepatic stem cells, hepatoblasts, pluripotent progenitors thereof, and progenitors committed to differentiate into a particular cell type (e.g., a hepatocyte or biliary cell). “Pluripotent” signifies cells that can form daughter cells of more than one fate; “unipotent” or “committed progenitors” are cells that have a single adult fate.

“Clonogenic expansion” refers to the growth property of cells that can expand from a single cell and be subcultured and expanded repeatedly with retention of the phenotype of the parent cell. “Colony formation” refers to the property of diploid parenchymal cells that can undergo a limited number of cell divisions (typically 5-7 cell divisions) within a week or two and involves cells with limited ability to undergo subculture or passaging.

Hepatic stem cells are pluripotent cells found in the ductal plates (also called limiting plates) in fetal and neonatal livers and in the Canals of Hering in pediatric and adult livers, show evidence of self-replication with expression of telomerase and are capable of forming mature liver cells when transplanted. These cells are EpCAM+, NCAM+, ALB+, CK8/18+, CK19+, CD133/1+, and are negative for all hemopoietic markers tested (e.g., CD34, CD38, CD45, CD14), mesenchymal cell markers (CD146, VEGFr, CD31) and for expression of P450s or alpha-fetoprotein. The hepatic stem cells have been found to give rise to hepatoblasts and to committed (unipotent) biliary progenitors.

Hepatoblasts (HBs) are also pluripotent cells found throughout the parenchyma of fetal and neonatal livers and as single cells or small aggregates of cells tethered to the ends of the Canals of Hering. HBs derive from the hepatic stem cells. HBs share many antigens present on HSCs but with important distinctions. For example, HBs do not express NCAM but rather ICAM1 and they express significant amounts of alpha-fetoprotein and fetal forms of P450s. These HBs give rise to the unipotent progenitors, the committed hepatocytic and biliary progenitors.

Hepatic Committed Progenitors are unipotent progenitors of either the hepatocytic and biliary lineages. Their antigenic profile overlaps with that of the HBs; however, biliary committed progenitors express CK19 but not AFP or ALB, whereas the hepatocytic committed progenitors express AFP and ALB but not CK19. Committed biliary progenitors derive directly from hepatic stem cells and also from hepatoblasts.

Mesenchymal Cells (MCs) include cells at various lineage stages of the many different mesenchymal cell types (listed as the mature cells and, in parentheses, their precursors): including stroma (mesenchymal stem cells), endothelia (angioblasts), stellate cells (stellate cell precursors), and various hemopoietic cells (hemopoietic stem cells).

While most, if not all, of the discussion and examples of hepatic progenitors herein will be with reference to human-derived cell populations, the teachings herein should not be limited to humans. In fact, one of ordinary skill in the art may be expected to apply the teachings herein to the expansion of hepatic progenitors from mammals, generally (e.g., mice, rats, dogs, etc.). Accordingly, the scope of the present invention is intended to include hepatic progenitors of any and all mammals.

Furthermore, while the invention has been described to a great extent to the isolation of hepatic progenitors from liver tissue. The methodology described herein is extends to the isolation of progenitor cells, not limited to hepatic progenitor cells, from other tissues, including pancreas, gut, lung, or bone marrow cells.

It is also noted that hepatic progenitors suitable for ex vivo propagation in accordance with the instant invention are not limited to those isolated or identified by any particular method. See for example U.S. Pat. Nos. 5,702,881; 5,660,976; 5,752,929; 5,863,296; 5,855,617; 5,843,024; 5,827,222; 5,723,282; 5,514,536; and 4,723,939 among many others and incorporated herein in their entirety by way of reference.

By way of specific example, methods for the isolation and identification of the hepatic progenitors have been described in, for example, U.S. Pat. No. 6,069,005 and U.S. patent application Ser. Nos. 09/487,318; 10/135,700; and 10/387,547, the disclosures of which are also incorporated herein in their entirety by reference. For example, hepatic stem cells and hepatoblasts share numerous antigens (e.g., cytokeratins 8, 18, and 19, albumin, CD133/1, and epithelial cell adhesion molecule (“EpCAM”) and are negative for hemopoietic markers (e.g., glycophorin A, CD34, CD38, CD45, CD14) and mesenchymal cell markers (e.g., CD146, CD31, VEGFr or KDR). Hepatic stem cells and hepatoblasts can be isolated by these markers.

Alternatively, hepatic stem cells and hepatoblasts can be distinguished from each other by size (the stem cells are 7-9 μm; the hepatoblasts are 10-12 μm), by morphology in cultures (the stem cells form dense, morphologically uniform colonies, whereas the hepatoblasts form cord-like structures interspersed by clear channels, presumptive canaliculi), by distinctions in the pattern of expression of certain antigens (EpCAM is expressed throughout the hepatic stem cells but is confined to the cell surface in the hepatoblasts), or by distinct antigenic profiles (N-CAM is present in the hepatic stem cells, whereas alpha-fetoprotein (AFP) and ICAM1 are expressed by the hepatoblasts).

In fetal and neonatal livers, the hepatic stem cells are in the ductal plates (also called “limiting plates”), whereas the hepatoblasts are the dominant parenchymal cell population (>80%). In pediatric and adult tissues, the hepatic stem cells are present in the Canals of Hering, whereas the hepatoblasts are cells tethered to the ends of the Canals of Hering. The hepatoblasts consist of small numbers of cells in normal tissue but found in large numbers (e.g., nodules) in diseased livers (e.g., cirrhosis).

Liver cell suspensions from fetal livers are replete with hemopoietic cells, especially erythroid cells. In fact, original cell suspensions of human fetal livers consist, on average, of only 6-9% parenchymal cells with the remainder being various non-parenchymal cells, particularly erythroid cells. As routine methods for elimination of erythroid cells, such as use of a lysis buffer, may be toxic for the hepatic progenitors, other methods may be preferable. For example, the erythroid cells may be separated from the parenchymal cells by repeated slow speed centrifugations using methods published previously (Lilja et al., 1997; Lilja et al., 1998).

An alternate and more efficient method, complement-mediated cytotoxicity, may be utilized to minimize the loss of candidate stem cells. After collagenase digestion, anti-human red blood cell (RBC) antibodies can be incubated with the cell suspension (1:5000 dilution) for 15 min at 37° C. To puncture and lyse antibody-marked erythrocytes, complement (e.g., LowTox Guinea Pig complement) is added (1:3000 dilution) for a 10 min incubation at 37° C. Cell supernatants become pinkish from hemoglobin released from erythrocytes. Suspensions purged of hemopoietic cells consist of at least 80-90% parenchymal cells.

The resulting suspension, from which the hemopoietic cells have been purged, is then subjected to a second round of enzymatic digestion in fresh collagenase solution for 30 min to minimize cell clumping, followed by sieving through a 75 μm nylon sieve. Estimated cell viability by trypan blue exclusion is routinely higher than 95%. After filtration, most hepatoblasts are clumped cells containing 4 to 8 cells per aggregate. Together these techniques help to generate cell suspensions substantially free of RBCs yet enriched parenchymal liver cells.

By way of another example, whole livers may be processed to remove the dead cells and hemopoietic cells as described in U.S. patent application Ser. No. 10/620,433. Briefly, whole human liver or resection thereof from neonatal, pediatric, juvenile, adult, or cadaver donor is perfused with a chelation buffer at approximately 37° C. for approximately 15 minutes and then digested with an enzyme preparation comprising collagenase and elastase at 37° C. for less than about 30 minutes to provide a digested liver. The digested liver is then chilled in a collection buffer and mechanically dissociated to provide a cell suspension. The cell suspension is then also filtered through a filter cartridge to remove debris and cell aggregates. The suspension of single cells is then collected and its viability and concentration is optionally determined. The concentration of the cells is adjusted to about 25 million cells per mL to provide a starting cell suspension. To remove dead cells from this suspension, an aliquot (250 mL) of the starting cell suspension is mixed with an equal volume of 25% iodixanol (OPTIPREP) solution in RPMI 1640 medium lacking phenol red. This mixture (500 mL) is overlaid with a predetermined volume (60 mL) of RPMI 1640 medium lacking phenol red and subjected to centrifugation on a COBE 2991 Cell Processor (15 min at 2000 rpm, ca. 500×g) to obtain at least one band enriched for viable cells. The band is collected into a third bag on ice. Optionally, the viability and concentration of cells may be determined.

Extracellular Matrix Components

Once a suspension of single liver cells is obtained, the present invention provides for the isolation or selection of hepatic progenitor cells (preferably hepatic stem cells) therefrom by plating the suspension on a gel-like extracellular matrix. In fact, without being limited to or bound by theory, the present inventors have found that the extracellular matrix components described herein are preferably used in fibrillar (i.e., polymerized) form. As defined herein, “polymerized” or “polymeric” collagen is synonymous with collagen in its fibrillar form. Of note, the definition, as well as the invention, is not limited by the extent of polymerization. In other words, any composition comprising a substantial portion of non-monomeric collagen is fibrillar. It is believed that when polymerized, the matrix proteins form a gel-like strata, which is conducive to hepatic progenitor isolation and propagation. The cells may be plated directly onto the matrix or “sandwiched” between two strata creating a 3D matrix.

To be sure, the scope of the present invention is not limited to any one matrix component or combination thereof. In keeping with the teachings herein, the present invention describes and teaches the use of any and all extracellular matrix components and their combination in the generation of substrata that can be utilized for ex vivo maintenance of cells either for expansion or for differentiation. While many of these components will be discussed below, for the sake of clarity, terminology such as type I, III and IV collagens and/or laminins, should be construed as mere representative of their respective class of extracellular matrix components.

In a specific embodiment of the present invention, a matrix comprising types I and III collagen is described. While many ratios of these collagens are suitable with the present invention, a preferred embodiment comprises 97:3 collagen 1:111. A solution of 97% collage type 1 and 3% collagen type III is commercially available from INAMED Corp. (Fremont, USA) under the mark PURECOL.

PURECOL is typically supplied at approximately 3 mg/mL and pH 2. The “solution” contains a high monomer content; however, the monomer may be polymerized according to the following manufacturer suggested protocol:

1. Mix 8 parts of chilled PURECOL collagen with 1 part of 10× phosphate buffered saline solution of 10× cell culture media. Cells can be added to the media at this step.

2. Adjust the pH of the solution to 7.4±0.2 by the addition of 0.1M NaOH or 0.01N HCl. The pH of the solution can be monitored by a pH meter or pH paper.

3. Maintain the temperature at 4-6° C. to prevent gelation.

4. To gel, warm the neutralized PURECOL collagen solution to 37° C. For best results, allow at least 45 minutes to an hour for gelation to occur.

5. The gels can be dried under a laminar flow hood.

For the experiments described herein, a PURECOL matrix was prepared 10×PBS to the PURECOL solution as-supplied. For example, in FIG. 1A, 1.0 ml of PURECOL consisting of 8 parts 1.5 mg/ml PURECOL and one part 10×PBS, pH 7.4 (using 0.1M NaOH or 0.1M HCl), was combined and gently mixed at 4° C. to generate a homogeneous solution. During this process, it is desirable to avoid introducing air bubbles within the newly formed collagen suspension as air gaps can destabilize the collagen. Preferably, varying ratios of human collagen types I and III as outlined in Table 1 are prepared as a gel as described above before applying to tissue culture plastic (TCP). After gelation, the plates/wells are ready to receive the cell suspensions.

As mentioned above, many ratios of these collagens are suitable with the present invention. In addition to those specifically recited above, other ratios include, but are not limited to the following listed in Table 1:

TABLE 1

| | % Type I | % Type III | % Other | % Non- |
|---|---|---|---|---|
| No. | Collagen | Collagen | Collagens¹ | Collagens² |
| 1 | 97 | 3 | 0 | 0 |
| 2 | 98 | 2 | | |
| 3 | 99 | 1 | | |
| 4 | 100 | 0 | | |
| 5 | 95 | 5 | | |
| 6 | 90 | 10 | | |
| 7 | 80 | 20 | | |
| 8 | 70 | 30 | | |
| 9 | 60 | 40 | | |
| 10 | 50 | 50 | | |
| 11 | 97 | 0 | 3 | |
| 12 | 97 | 0 | | 3 |
| 13 | 97 | 1 | 2 | 0 |
| 14 | 97 | 1 | 1 | 1 |
| 15 | 97 | 0 | 3 | 0 |
| 16 | 95 | 1 | 3 | 1 |
| 17 | 95 | 0 | 0 | 5 |
| 18 | 95 | 0 | 5 | 0 |

¹Other collagens include, for example, type IV collagen
²Non-collagens include, for example, fibronectin

Without being held to or bound by theory, it is believed that a greater percentage of collagen I to collagen III favors progenitor cell enrichment, and moreover, that the gel (i.e., fibrillar) format of collagen as opposed to the film (i.e., monomeric) format can drive progenitor cell enrichment.

Optional Enrichment Steps

While not required with the instant invention, further enrichment protocols either prior or subsequent to plating a suspension of whole liver cells onto the matrices described herein include Ficoll fractionation for isolation of hepatoblasts. Briefly, cells are suspended in 10 ml of basal medium without phenol red and overlayed onto an equal volume of Ficoll-Paque (Amersham Pharmacia) in a 50 ml centrifuge tube. Cells are subsequently centrifuged at 1000×g for 25 minutes. Both the interface and the pelleted cells are collected. Ficoll fractionation yields pellets that are generally more than 80% parenchymal cells, essentially all of which are hepatoblasts, and an interface of 13-14% of the original cell population with diverse antigenic profiles indicating parenchymal, hemopoietic and endothelial cells. The Ficoll interface cells yield colonies of ductal plate cells at 0.1%, a 10-fold increase over that from the original cell suspension. While results with Ficoll fractionation may be consistent for isolation of hepatoblasts, they tend to be more variable from prep to prep in the isolation of the stem cells.

Immunoselection is another technique for enrichment and/or isolation of hepatic stem cells (ductal plate cells) or other subpopulations. Preferable immunoselection protocols are those which employ antigens with intense expression on cells (e.g., EpCAM found on all hepatic progenitors) and others on one subpopulation of hepatic progenitor (e.g., NCAM on ductal plate cells). Immunoselection can be by any of the diverse methods for these procedures and can be cytometers, panning, or magnetic beads.

Magnetic Immunoselection comprises the isolation of cells expressing, for example, EpCAM from human liver cell suspensions using monoclonal antibody HEA125 coupled to magnetic microbeads, and an autoMACS™ or CliniMACS® magnetic column separation system from Miltenyi Biotec (Bergisch Gladbach, Germany), following the manufacturer's recommended protocols. Similar methods were used for immunoselection of NCAM, CD146, KDR (VEGFr), and CD133/1 cells.

Media & Buffers

A variety of cell culture media may be appropriate with the instant invention. By adding or removing growth and/or differentiation factors from the culture medium, the rate of cell proliferation and/or differentiation may be influenced. For example, the addition of serum can slow growth of the hepatic progenitors and cause lineage restriction towards the hepatocytic fate and, in parallel, cause rapid expansion of mesenchymal cell populations (stroma and endothelia). The addition of epidermal growth factor leads to lineage restriction towards an hepatocytic fate.

Preferably, in some embodiments, the matrix components described herein are employed in combination with a serum-free medium. A serum-free media was developed previously for hepatoblasts and is described in U.S. patent application Ser. No. 09/678,953, the disclosure of which is incorporated by reference herein in its entirety. Without being held to or bound by theory, it is presently believed that that matrix components of the present invention provide many of the survival, growth and/or proliferation signals generally provided by feeder cells. Thus, the instant invention may replace, in significant part, the need for embryonic stromal feeder cells to maintain viability and expansion potential of the hepatic progenitors.

Unless otherwise noted, serum free media was used during processing of the liver tissue and maintenance of cell cultures. This media comprises 500 ml RPMI 1640 supplemented with 0.1% bovine serum albumin, Fraction V, 10 μg/ml bovine holo-transferrin (iron saturated), 5 μg/ml insulin, 500 μl selenium (5×10⁻⁵ M stock), 5 ml L-glutamine, 270 mg niacinamide, 5 ml AAS antibiotic, 500 μl hydrocortisone (10⁻⁴ M stock), 1.75 μl 2-mercaptoethanol and 38 μl of a mixture of free fatty acids prepared as published in U.S. patent application Ser. No. 09/678,953. This medium is further supplemented with 0.2 to 1 unit/mL of LIF (ES-GRO; Chemicon, Inc., Temecula, Calif.), 0-10 ng/ml BMP4 (R & D Systems), and 0-20 μM PD098059 (Mek inhibitor, Upstate, Lake Placid, N.Y.). Finally, the media is sterilized and its pH adjusted to 7.4 prior to use.

“Cell Wash Buffer” comprises 500 ml RPMI 1640 supplemented with 1% bovine serum albumin, 500 μl selenium and 5 ml of AAS antibiotic. “Enzymatic Digestion Buffer” comprises 100 ml Cell Wash Buffer supplemented with 60 mg type IV collagenase and 30 mg DNase dissolved at 37° C.

Proliferation

Identification and documentation of proliferation of hepatic progenitors was assessed macroscopically by imaging size changes of colony growth using phase microscopy with 4×, 10×, and 20× magnifiers; the low magnification objectives allowed observation of entire colonies. Progenitor colonies were comprised of tightly adherent cells approximately 7-10 μm in diameter. Growth curves were obtained by repetitive colony imaging, and the micrographs normalized against known dimensions precalibrated on MetaMorph Image Software for statistical comparative analysis. Alternatively, proliferation of hepatic progenitors was quantitated using either the CellTiter 96 Non-radioactive cell proliferation assay (MTT) or One solution cell proliferation assay (MTS) (Promega, Madison, Wis.) as recommended by the manufacturer.

Typically, the period of growth required for the enrichment of hepatic progenitors cells in accordance with the invention ranges from 6 days for fetal parenchymal cell populations, 10 days for neonatal cell parenchymal populations to 60 days for adult parenchymal cell populations.

Tissue Acquisition & Preparation

Liver tissue from human fetuses between 16-22 weeks gestational age were obtained from an accredited agency, Advanced Biosciences Resources of Alameda, Calif. Preferably, tissues were received within 18 hours of isolation and arrived as multiple sections of liver tissue or, on occasion, as reasonably intact liver. In general, overall tissue volumes ranged between about 4 ml and about 12 ml and contained large quantities of red blood cells (RBCs).

The livers were mechanically dissociated, and the tissue was partially digested with the enzymatic digestion buffer yielding clumps of parenchymal cells. These clumps were subjected to washing and low speed centrifugation to substantially eliminate free floating hemopoietic cells yet retain hepatic parenchyma. The dissociated livers were then segmented into 3 ml aliquots to which 25 mls of Enzymatic Digestion Buffer was added. After 30 minutes of moderate agitation at 32° C., the supernatant was removed and stored at 4° C. Any residual unfragmented pellets were re-digested with fresh Enzymatic Digestion Buffer for an additional 30 minutes. After completion of the enzymatic digestion of the tissue fragments, the cell suspensions were centrifuged at 250 revolutionary centrifugal force (RCF); the supernatants removed; and the pellets resuspended in an equivalent amount of Cell Wash Buffer.

Immunostaining

Immunostaining of cells was done after culture fixation using a 50/50 mixture of acetone and methanol for 2 minutes, washed again with 1×PBS, and blocked with 10% goat serum for 45 minutes. Then a primary human antibody, conjugated with a fluorescent probe, was added for 1 to 8 hours at room temperature. When unconjugated primary antibody was used, the cells were stained with a secondary antibody conjugated with a fluoroprobe.

Embodiment of the instant invention will now be described by way of non-limiting examples.

### EXAMPLES

Several extracellular matrices were used as substrates to plate single cell suspensions of whole liver (e.g., “UMIX”) or EpCAM+ magnetic sorted cells (i.e., hepatic stem cells). The matrices included (a) untreated; (b) PureCOl™; (c and d) collagen III [from Sigma-Aldrich or Becton-Dickinson (BD), respectively]; (e) collagen IV (BD), (f) collagen I (Biocoat; Becton-Dickinson); or (g) fibronectin (Sigma). PureCol™ (Inamed) is comprised of 97% collagen I and ˜3% collagen III in a gel format (fibrillar form). Collagen III from both Sigma-Aldrich and Becton-Dickinson is comprised of primarily collagen III with a minor percentage of collagen I in a film format. Collagen IV is in a film format and Collagen I from Becton-Dickinson is also provided in a film format (monomeric form). Fibronectin (Sigma-Aldrich, Inc, St. Louis, Mo.) plates were prepared at 5 μg/cm² and adjusted to pH 7.5. Collagen III and IV plates were prepared at concentrations of 1.0 mg/ml.

### Example I

Neonate—37 weeks gestation. 1 billion cells were used for immunoselection of EpCAM+ hepatic progenitor cells. The neonatal EpCAM-sorted cells were plated in (a) untreated tissue culture plastic (TCP); (b) PURECOL; and (c-d) Collagen III (Sigma and BD, respectively) at 100,000 or 300,000 cells per well in a 6-well dish. Stem cell colonies were obtained in the PureCol plates two weeks post-plating, whereas no stem cell colonies were obtained in any of the other conditions.

### Example II

Neonate—38 weeks gestation. 7.3 billion cells were recovered, nearly all of which were immunoselected for EpCAM+ hepatic progenitor cells. The neonatal EpCAM-sorted cells were plated in (a) untreated tissue culture plastic (TCP); (b) PURECOL; and (c-d) Collagen III (Sigma and BD, respectively) at 800,000 cells per well in a 6-well dish. In addition, the non-immunoselected whole cell population, comprising both parenchymal and nonparenchymal cells, was also plated at 800,000 cells per well (6-well dish) on (a) untreated TCP; (b) PURECOL; and (c-d) Collagen III (Sigma and BD, respectively) plated at confluency for UMIX and 800,000 cells per well for EpCAM sorted cells in 6-well dishes.

Within two weeks of ex vivo propagation, stem cell like colonies were observed (FIG. 1). The photomicrographs shown in FIG. 1 were obtained after the two weeks of culture of UMIX cells plated at equal cell densities. The PURECOL propagated culture was photographed at lower magnification to better illustrate the extensive growth of the putative hepatic stem cells. Hepatic stem cells form compact dense aggregates of cells which are 7-10 μM in diameter. In untreated and collagen III treated plated, the UMIX cell cultures did not yield any stem cell colonies though fibroblast-like cells were observed. Similar results were obtained using EpCAM-sorted cells. Interestingly, the EpCAM sorted cells plated on PURECOL also yielded stem cell colonies; however, the non-immunoselected population yielded a higher stem cell enrichment. None of the other plating conditions yielded any stem cell colonies.

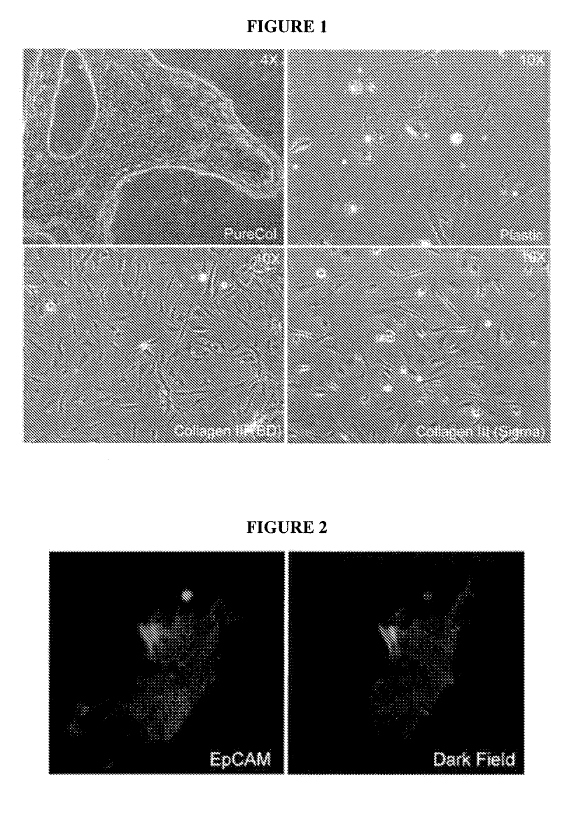

Two weeks post-plating cultures were fixed and stained for EpCAM. All of the cells stained positive for EpCAM. For a negative control, parallel cultures were visualized without staining with secondary antibodies; no signal was observed. Thus, the PURECOL matrix alone can be used to select hepatic stem cells from whole liver cells (see FIG. 2).

### Example III

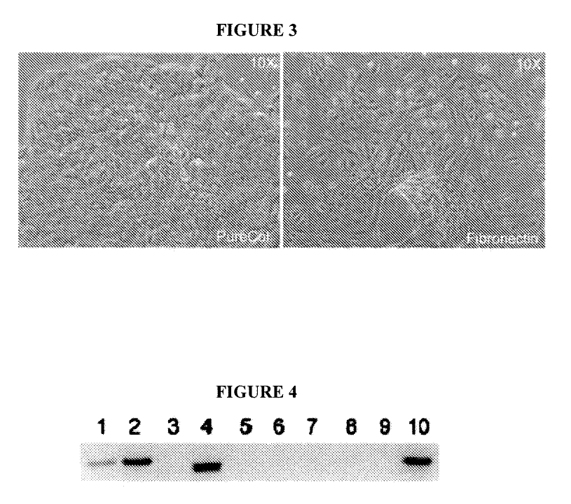

Fetal liver cells were plated at 800,000 cells per well in 6-well dishes on (a) untreated TCP; (b) PURECOL; and (f) Biocoat Collagen I. Five weeks post-plating conditions (a) and (f) yielded less than 1% hepatic stem cells as determined by morphology identical to that shown in FIGS. 1 and 3—PURECOL panel). Condition (a) yielded >50% hepatic stem cells, however, the cells ceased to proliferate after 35 days.

### Example IV

Fetal liver cells were plated at 800,000 cells per well in 6-well dishes on (a) untreated TCP at 4.6×10⁶ cells in 10 cm dishes; and (b) PURECOL. Four weeks post-plating, condition (a) yielded less than 2% hepatic stem cells, while condition (b) yielded >40% hepatic stem cells.

### Example V

Fetal liver cells were plated at 800,000 cells per well in 6-well dishes on (a) untreated TCP; (b) PURECOL; (c,d) collagen III (Sigma-Aldrich and BD, respectively); (e) collagen IV, (f) collagen I (Biocoat; Becton-Dickinson); and (g) fibronectin. One week post-plating, conditions (a) and (c-g) yielded less than 2% hepatic stem cells, while condition (b) yielded >40% hepatic stem cells (see FIG. 3).

Taken together, these experiments demonstrate that single cell suspensions of whole liver can be substantially enriched for hepatic progenitor cells, preferably hepatic stem cells, when cultured on a gel-like extracellular matrix, preferably comprising fibrillar collagens, more preferably fibrillar collagen I. In some embodiments of the invention, substantial enrichment is defined at greater than about 40%, 50%, or 60% enrichment over whole liver cell populations. In preferred embodiments, enrichment is greater than about 70%, 80%, or even 90% enrichment over whole liver cell populations.

As documented above, in other embodiments of the present invention, single cell suspensions of whole liver can be substantially isolated or selected for hepatic progenitor cells, preferably hepatic stem cells, when cultured on a gel-like extracellular matrix, preferably comprising fibrillar collagens, more preferably fibrillar collagen I. In these embodiments, isolation yields populations of greater than about 90%, preferably greater than about 95%, and most preferably, greater than about 99% “pure” populations of hepatic progenitors cells, preferably hepatic stem cells.

Cultures of stem cell colonies were typically obtained within two weeks after liver processing. For the fetal preparations, highly enriched stem cell-like colonies were visible one week after liver processing. Similar results could be found with adult liver cell populations.

Again, the data show that stem cells expand and maintain better on a matrix consisting predominately of collagen I in a fibrillar format, and that this matrix provides a simple selective medium to obtain highly enriched populations (approaching if not achieving 100% enrichment) of hepatic stem cells from neonatal and fetal whole liver cell preparations. In other words, the inventive method allows for the selection of hepatic stem cells without any other criteria. For example, the instant method obviates the need for sorting for EpCAM+ cells.

RNA from these cultures could be used to perform RT-PCR analyses for a number of biomarkers (e.g., EpCAM (expressed in stem cells), ALB (expressed in stem cells), AFP (expressed in hepatoblasts but not stem cells), CYP3A4 (expressed in mature hepatocytes), CK19 (expressed in stem cells and biliary cells), and GAPDH (control for input RNA quality and internal standard for RNA quantities) (FIG. 4). To be sure, hepatic stem cells are known to be EpCAM+AFP-ALB+. In this way, cells can be identified as “stem cell,” “hepatoblast,” or “mature hepatocytes.”

The novel findings herein enable the transplantation of cells or a population of cells and may obviate the need for whole organ replacement all together. Indeed, the inventive compositions of the present invention could be used for repopulating diseased livers. Whole liver cell preparations from either fetal, neonatal, pediatric, or adult donor organs, or already enriched stem cells from these sources may be plated at densities of 800 to 850 cells/cm² (3.5−4.0×10⁶ cells per 150 mm dish) on a novel matrix as described herein (e.g., collagen I and III gel (33:1 ratio)). Upon stem cell enrichment, over 3×10⁷ cells per 150 mm dish can be obtained. Generating the 3×10⁹ cells or 1% of liver mass that can be safely transplanted into a patient would require about one hundred 150 mm dishes (utilizing a total of 3.5−4.0×10⁸ liver cells; an adult liver contains over 50×10⁹ cells).

At this point, the cells can be utilized for cell transplantation, cryopreserved for future expansion, or expanded further by removing the cells from the 150 mm dish by trypsin or collagenase digestion and plated at a 1:10 dilution (i.e., expand one dish to ten dishes). Serum-free medium supplemented with factors such as combinations of LIF, EP0, PD98059, BMP4, and choline, that support robust stem cell proliferation will be used to culture the stem cells.

For products intended for human use, all processing steps should preferably be conducted according to cGMP standards with appropriate SOPs. The human stem cells should be isolated by trypsin or collagenase digestion. Cells should be used immediately or cryopreserved prior to use. The human stem cells, if cryopreserved, may be thawed and suspended in a serum free, iso-osmotic media suitable for infusion at a preferred concentration of no less than 0.1 and no more than 10 million cells per ml. Cell viability could be assessed by tryphan blue exclusion or equivalent assay. Cells may be stored at room temperature or on wet ice and assessed by microscopic examination for evidence of clumping. If clumping is observed, the cells would be subjected to dispersion over a nylon mesh (40 micron) filter and the final concentration of cells re-determined.

Cells may be infused via the hepatic portal vein or intrasplenically into a recipient suffering from liver damage due to acute toxin exposure at a preferred dose of 1 to 10 million cells per kg of body weight. As an alternative to cell infusion, the stem cells may be embedded into any of variety of scaffolds (molds) for grafting directly into the liver using the same dosage parameters outlined above. Recipients should be monitored for evidence of any acute reactions, such as fever, chills, or change in mental status, and if any of these symptoms are observed, should be treated symptomatically by standard medical practice. Subjects are then monitored over the following 2 month period by weekly serum chemistries to assess liver function recovery.

In other embodiments, in vitro devices such as bioreactors may be seeded with hepatic progenitors enveloped in an appropriate extracellular matrix and soluble signaling environment so they populate device subcompartments with viable tissue structures. In this way, bioartificial livers are developed as extracorporeal liver assist devices to support patients in organ failure. They may also be used as adjuncts to transplantation of liver cells to enable a patient to have liver function even while transplanted donor cells are reconstituting normal liver tissue. Clinical trials have been completed or are ongoing using cell lines (e.g., porcine liver cells and human liver cells).

The inventive compositions of the present invention could be used for seeding liver assist devices with stem cells as follows: whole liver cell preparations from either fetal, neonatal, pediatric, or adult donor organs, or enriched stem cells from these sources are plated at densities of 800 to 850 cells/cm² (cell number will depend on the volume capacity of the liver assist device) on a matrix in accordance with the instant invention, preferably, collagen I and III gel (33:1 ratio). Cell culturing will result in the generation of a highly enriched population of hepatic stem cells within the liver assist device. Serum-free medium supplemented with factors, including combinations of LIF, EP0, PD98059, BMP4, and choline, that support robust stem cell proliferation will be used to culture the stem cells.

Bioartificial devices can also be utilized for pharmacology studies, vaccine development, and as a bridge between organ failure and organ transplantation. Hundreds of candidate pharmaceutical compounds are synthesized each year and the cost of animal testing, (which may exceed several million dollars to test a single substance for safety assessment) needs to be reduced. The development of in vitro model systems to evaluate the toxicity of chemicals and drugs has thus become increasingly important. These in vitro systems may also enhance understanding of the mechanisms of drug- and chemical-induced toxicity. In vivo models are complicated by the presence of structural and functional heterogeneity of biochemical pathways and do not allow for mechanisms to be clearly defined or reproducibly examined. The current technology for testing drug candidates is based on two-dimensional (2-D) sandwich cell culturing techniques developed four decades ago.

The recent discovery that rat hepatocytes maintain elevated levels of metabolic function for at least 14 days in the multicoaxial bioreactor (MCB) has demonstrated the proof-of-principle of the MCB. The inventive compositions of the present invention could be used for seeding MCBs for in vitro drug- and chemical-induced toxicity studies. This novel product containing hepatic stem cells has the potential to save the pharmaceutical industry significant time and financial resources by identifying idiosyncratic drug reactions that would not have been revealed using current 2-D techniques.

Indeed, the results obtained from these investigations suggest that utilizing these cells may be an avenue to improve cell sourcing limitations that currently inhibit both cell therapy and bioreactor device medical treatments options. In accordance with the teachings described herein, whole liver cell preparations from either fetal, neonatal, pediatric, or adult donor organs, or enriched stem cells from these sources are plated at densities of 2×10⁶ cells per ml volume (cell number will depend on the volume capacity of the bioreactor) on a matrix in accordance with the instant invention, preferably, collagen I and III gel (33:1 ratio). Cell culturing will result in the generation of a highly enriched population of hepatic stem cells within the liver assist device. Serum-free medium supplemented with factors, including combinations of LIF, EPO, PD98059, BMP4, and choline, that support robust stem cell proliferation will be used to culture the stem cells.

While the invention has been described in connection with specific embodiments thereof, it will be understood that it is capable of further modifications and this application is intended to cover any variations, uses, or alterations of the invention following. In general, the principles of the invention and including such departures from the present disclosure as come within known or customary practice within the art to which the invention pertains and as may be applied to the essential features hereinbefore set forth and as follows in the scope of the appended claims.

What is claimed is:

1. A method of isolating hepatic progenitors in vitro comprising:
(a) providing a single cell suspension of hepatic cells;
(b) culturing the suspension of hepatic cells on an extracellular matrix comprising a collagen in polymerized form to obtain a population of isolated hepatic progenitor cells.

2. The method of claim 1 in which the collagen is a type I collagen.

3. The method of claim 2 in which the matrix comprises greater than about 75 percent by weight type I collagen.

4. The method of claim 3 in which the matrix comprises greater than about 90 percent by weight type I collagen.

5. The method of claim 4 in which the matrix comprises greater than about 95 percent by weight type I collagen.

6. The method of claim 5 in which the matrix comprises greater than about 97 percent by weight type I collagen.

7. The method of claim 1 in which the matrix further comprises a type III collagen in polymerized form.

8. The method of claim 1 in which the hepatic progenitors are hepatic stem cells.

9. The method of claim 1 further comprising culturing the suspension of hepatic cells in serum free culture medium.

10. The method of claim 1 in which the extracellular matrix comprising a collagen in polymerized form is a PURECOL matrix.

11. A method of selecting hepatic progenitors in vitro comprising:
(a) providing a single cell suspension of hepatic cells;
(b) culturing the suspension of hepatic cells on an extracellular matrix comprising a collagen, in which the collagen is in polymerized form, to obtain a population of isolated hepatic progenitor cells.

12. A method of selecting hepatic progenitors in vitro comprising:
(a) providing a single cell suspension of hepatic cells;
(b) culturing the suspension of hepatic cells on an extracellular matrix comprising a collagen, in which the collagen is in polymerized form, to obtain a population of isolated hepatic progenitor cells.

13. A composition comprising a cell culture of hepatic progenitor cells, serum-free culture medium, and an extracellular matrix comprising a collagen in polymerized form.

14. The method of claim 13 in which the collagen is a type I collagen.

15. The method of claim 13 in which the matrix comprises greater than about 95 percent by weight type I collagen.

16. The method of claim 15 in which the matrix comprises greater than about 97 percent by weight type I collagen.

17. The method of claim 13 in which the matrix further comprises a type III collagen in polymerized form.

18. The method of claim 13 in which the hepatic progenitors are hepatic stem cells.

19. A method of propagating hepatic progenitors in vitro comprising:
(a) providing a single cell suspension of hepatic cells;
(b) culturing the suspension of hepatic cells on an extracellular matrix comprising a collagen and in serum-free culture medium, in which the collagen is in polymerized form.

20. The method of claim 19 in which the hepatic progenitors are isolated hepatic stem cells, isolated hepatoblasts, committed hepatic progenitors, or a combination thereof.

21. The method of claim 20 in which the hepatic progenitors are obtained from adult liver.

22. The method of claim 21 in which the adult liver is adult human liver.

23. The method of claim 19 in which the extracellular matrix further comprises a laminin.

24. The method of claim 1 in which the type I collagen is at a concentration between about 0.1 to about 15 μg/cm².

25. A container for propagation of hepatic progenitors comprising:
(a) a container, and
(b) an insoluble material comprising at least one collagen in polymerized form;
wherein the insoluble material substantially coats at least one surface of the container.

26. The container of claim 25 in which the container is a tissue culture plate, a bioreactor, a lab cell or a lab chip.
